(19) United States
(12) Patent Application Publication
(10) Pub. No.: US 2009/0010991 A1
(43) Pub. Date: Jan. 8, 2009

(54) Nasal Passage Stent

(76) Inventors: Julian Prabhu, Huntington Beach, CA (US); Luan M. Prabhu, Mississauga (CA); Sean C. Prabhu, Mississauga (CA); Andre J. Prabhu, Mississauga (CA)

Correspondence Address: JULIAN PRABHU, 17372 WILDROSE LN., HUNTINGTON BEACH, CA, 92649, US

(21) Appl. No.: 12/131,262

(22) Filed: Jun. 2, 2008

Related U.S. Application Data
(60) Provisional application No. 60/948,447, filed on Jul. 7, 2007.

(51) Int. Cl. A61K 9/00; A61P 43/00

(52) U.S. Cl. 424/424

(57) ABSTRACT

A drug coated nasal stent implant that provides filtration of environmental pollutants and airborne allergens from inhaled air regulating the air before it is ingested into the remainder of the respiratory tract. The coatings include but are not limited to a variety of allergenic-specific antigen targeted antibodies as well as anti-inflammatory drugs such as antihistamines

### BRIEF SUMMARY OF THE INVENTION

A nasal implant that comprises a stent, made of stainless steel or similar material, plastic or similar material, nano or similar material that is expandable from a closed to an open position (may or may not be drug coated) and a removable filter (may or may not be drug coated) which is threaded or press fit into the stent and filters any environmental pollutants/viruses/bacteria/chemicals etc.) from the air before it is ingested thru the nasal passage into the trachea/lungs.

### FIELD OF INVENTION

This invention relates to the medical field, affecting humans. In particular, the invention relates to the nasal cavity leading to the lungs providing patient's with regulated purified air.

### BACKGROUND

Various external nasal devices may exist that enhance the looks of a patient or guide their breathing ability. Generally patient's or users can undergo nasal surgery (internal or external), cosmetic surgery, breathing strips etc., that allow the patient or user to breathe easier. However, to date, there is no known commercial internal nasal device that purifies the environment air before it enters the nasal cavity. Millions of patient's worldwide suffer from environmental pollution (outdoor/indoor, viruses, chemicals, pollen etc.) and often resort to over the counter medication/s or surgery to experience relief. The use of medications, surgery can cause enormous financial strain on individuals, insurance companies, hospitals etc.

As a result there exists a great need for a nasal implant device that can provide regulated cleaner purified air to a patients lungs, enhancing the patient's immune system thereby greatly increasing the quality of a patient's life and reducing the financial burden on their families, insurance companies and hospitals.

### BRIEF DESCRIPTION OF SEVERAL VIEWS OF DRAWINGS

Having thus described this invention in general terms, reference is now being made to the drawings (not to scale).

FIG. 1 shows a side view of the collapsible design stent in the open and closed position.

FIG. 2 shows a side view of the filter support, whose primary function is to hold the stent and filter in place in the nostril.

FIG. 3 shows a side view of the carbon, nano, HEPA or similar filter that may be threaded or press fit.

FIG. 4 shows a side view of the integrated stent with the filter support and filter threaded or press fit into the stent.

### DETAILED DESCRIPTION OF THE INVENTION

With reference to the accompanying drawings, this invention process will be described fully hereinafter, in which some but not all embodiments of the invention are shown. The invention may be embodied in many different forms and should not be considered as limited to the embodiments set forth herein.

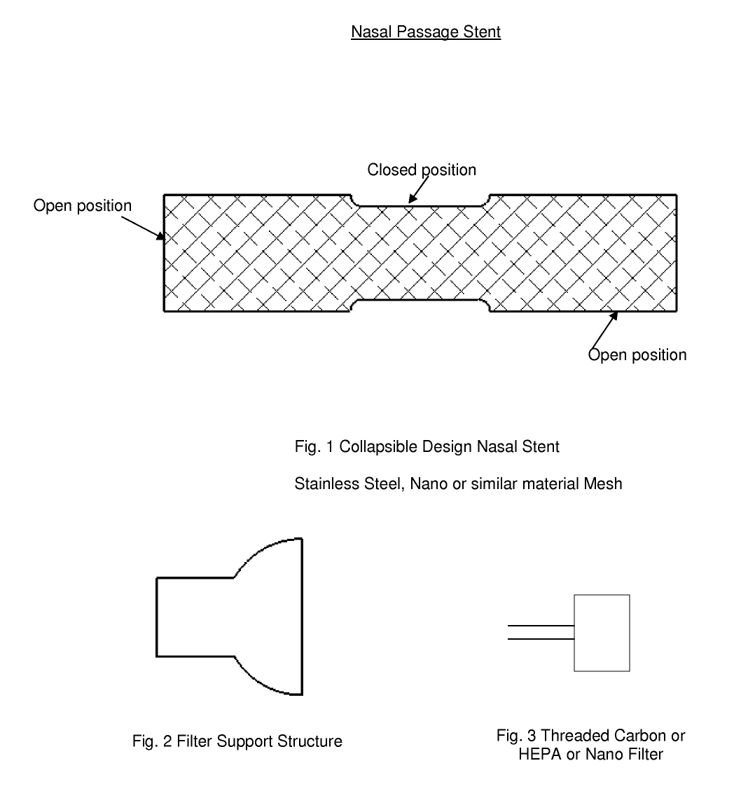

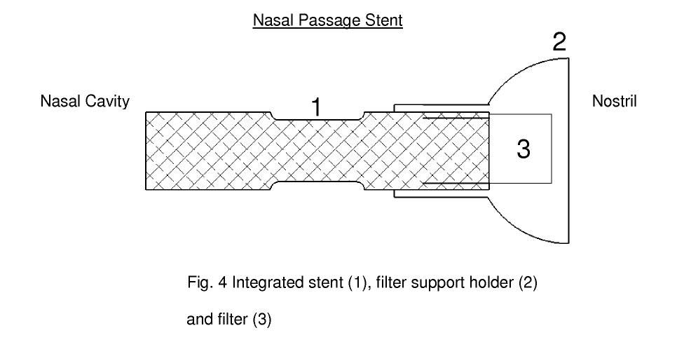

This invention is a stent made from a plastic or stainless steel mesh or similar like materials (FIG. 1). This device is implanted in the nasal cavity at the bone structure with the frontal end protruding into the nostril and the rear end protruding into the nasal cavity. Once inserted, the stent is expanded to the open position as shown in FIG. 1, thereby locking it in position and securing it permanently to prevent it from dislodging into the nasal passage/trachea/lungs. The frontal portion of the stent is press fitted with a shroud or filter support as shown in FIG. 2. The filter, which is made of carbon, or a HEPA filter as shown in FIG. 3, is threaded into the collapsible stent as a press fit into the filter support. The complete integrated assembled device as shown in FIG. 4 may or may not be coated with appropriate drugs to suit a patient's symptoms. The integrated assembled device provides for regulated breathing and purified environmental air before it is ingested into the lungs through the nasal cavity. In the case of a drug-coated stent, the drugs may include, but are not limited to allergenic antigen-specific antibodies as well as antihistamines. The antibodies used may be those in which their constant (or FC) regions are modified such that they are unable to bind to mast cells etc., and thus unable to promote an immune response.

What is claimed is:

1. A stent that is implanted or installed into the nasal cavity or nasopharynx through the nostrils or through nasal surgery.

2. With ref. to 1 and FIG. 1, what is claimed is the stent, which is a collapsible design mesh or similar design made from stainless steel or similar materials.

3. With ref. to 1, what is claimed is the stent, which is a collapsible design mesh or similar design made from plastic or similar materials.

4. With ref. to 1, what is claimed is the stent that is threaded or a press fit design, which is a collapsible design mesh or similar design made from nano materials.

5. With ref. to 1, what is claimed is the filter support conical holder made from stainless steel or similar metals or pliable plastic materials.

6. With ref. to 1, what is claimed is the filter that is threaded or press fit into the collapsible stent.

7. With ref. to 1, what is claimed is the stent and filter may be of various shapes, such as square or round or rectangular in nature.

8. With ref. to 1, what is claimed is that the stent may be drug coated with one or more drugs.

9. With ref. to FIG. 1, what is claimed is the filter may be drug coated with one or more drugs.

10. With ref. to 8, what is claimed is that the drugs may or may not be administered through a time-release mechanism.

11. With ref. to 8 and 9, what is claimed is that the particular combination of drugs administered to a patient may be custom tailored to the particular allergies of the patient.

12. With ref. to 8 and 9, what is claimed is that the drugs may include but are not limited to anti-inflammatory agents such as anti-histamines.

13. With ref. to 8 and 9, what is claimed is that the drugs may include but are not limited to antibodies for allergenic-specific antigens.

14. With ref. to 8 and 9, what is claimed is that the drugs may include vaccinations for various infections and conditions.

15. With ref. to 13, what is claimed is that the allergenic-specific antigens may include but are not limited to environmental pollutants, the pollen of various allergenic plants and the dander of various animals (including, but not limited to, cats and dogs).

16. With ref. to 13, what is claimed is that the antibodies used may be modified in ways that include, but are not limited to, altering the constant (or mast cell binding) domain of the molecular structure such that they are rendered unable to bind to circulating or stationary mast cells of the human immune system.

17. With ref. to 1, what is claimed is the stent and filter may be drug coated with one or more drugs to treat allergies, asthma and other respiratory ailments. Pharmaceuticals dispensed by the medicated stent may include but not be limited to the following: glucocorticoids, leukotriene modifiers and mast cell stabilizers.
